(19) United States
(12) Patent Application Publication
(10) Pub. No.: US 2012/0045771 A1
(43) Pub. Date: Feb. 23, 2012

(54) METHOD FOR ANALYSIS OF NUCLEIC ACID POPULATIONS

(75) Inventors: Markus Beier, Weinheim (DE); Peer F. Staehler, Mannheim (DE); Cord F. Staehler, Weineim (DE); Daniel Summerer, Mannheim (DE); Jack T. Leonard, South Hamilton, MA (US); Stephan Bau, Darmstadt (DE); Anthony Caruso, Harvard, MA (US); Nadine Schracke, Hirschberg (DE); Andreas Keller, Puettlingen (DE); Helmut Hanenberg, Duesseldorf (DE); Olaf Eckermann, Duesseldorf (DE)

(73) Assignee: FEBIT HOLDING GMBH, Heidelberg (DE)

(21) Appl. No.: 13/139,320

(22) PCT Filed: Dec. 11, 2009
(86) PCT No.: PCT/EP09/66945

§ 371 Date: Oct. 27, 2011

(30) Foreign Application Priority Data
Dec. 11, 2008 (DE) 10 2008 061 772.5
Dec. 11, 2008 (US) 61121621

(51) Int. Cl. C12Q 1/68; C07H 21/00; C12S 3/20

(52) U.S. Cl. 435/6.14; 435/6.1; 435/6.15; 435/270; 536/25.41

(57) ABSTRACT

The invention relates to a method for isolation of target molecules from a nucleic acid population.

The invention relates to a method for isolation of target molecules from a nucleic acid population.

With the aid of the so-called next generation sequencing methods (NGS), it is possible to sequence large sections of a genome with massive parallelity. However, since the number of base information thereby obtained is still considerably smaller in order to determine with it a complex eukaryotic genome, e.g. the genome of a human, mouse or rat, completely, at least in simple sequence coverage, enrichment methods are used in order to be able to analyze the medically/diagnostically interesting part regions of these genomes with NGS. Often, however, it is desirable to generate medically relevant data for a large number of individuals at reasonable cost for statistical reasons. Focusing on smaller regions of interest therefore allows to generate relevant statistical data from large populations.

The present invention provides processes and methods for making possible a focused analysis of medically relevant parameters in a large number of genomes.

Methods for enrichment of desired target molecules in a nucleic acid population based on a solid matrix (e.g. microarrays, beads) or a liquid matrix (nucleic acid libraries in solution) exist. Enrichment methods by means of a large number of PCRs performed in parallel are furthermore also known. Such methods are described e.g. in U.S. Pat. No. 6,013,440, U.S. Pat. No. 6,632,611, U.S. Pat. No. 7,214,490, DE 101 49 947 and U.S. Pat. No. 7,320,862, WO 2007/057652, WO 2008/115185, US 2008/194413, P. Parameswaran, Nucleic Acid Research, 2007, 35(19), e130, M. Meyer, Nucleic Acid Research, 2007, 35(15), e97, E. Hodges, Nature Genetics, 2007, 39(12):1522-7, T. Albert, Nature Methods, 2007, 4(11):903-5, or D. W. Craig, Nat Methods, 2008 October; 5(10):887-93.

The aim of the invention is to provide novel methods and uses in order to make possible an effective analysis of medically relevant genomic parameters.

The invention provides the analysis of population mixtures of nucleic acids. The invention therefore relates to methods for isolation of target nucleic acid molecules comprising the steps:
(a) providing a mixture of at least two populations of nucleic acid molecules,
(b) bringing the mixture into contact with a population of capture molecules under conditions under which target nucleic acid molecules from at least one of the populations can bind specifically to the capture molecules,
(c) separating off material not bound to capture molecules and
(d) isolating and optionally characterizing the target nucleic acid molecules isolated.

Preferred uses of the present invention are:
1) Sequence comparison
2) Mutation analysis
3) SNP detection
4) Exon junction analysis
5) Analysis of translocations, in particular in the context of tumor diagnostics
6) Analysis of variations in the number of copies
7) Pathogen detection
8) Detection of viral integration sites in a host genome and
9) Recursive Walking.

The present invention makes it possible to isolate from complex mixtures of nucleic acid populations target molecules, i.e. subpopulations, of interest or the corresponding content of interest of the nucleic acid population, and to make these available for sequence analysis. The target molecules can contain known and/or unknown sequences, e.g. mutations, SNPs, deletions, insertions, etc. The target molecules can be characterized by conventional sequencing technologies (Sanger technology, capillary sequencing) or also by the latest high throughput methods (Next Generation Sequencing=NGS) or also by other methods of sequence determination (pyrosequencing, microarrays etc. that are known to the person skilled in the art).

Nucleic acid populations are complex nucleic acid mixtures that can be of natural or artificial origin. The nucleic acid populations can be DNA or RNA or mixtures thereof. They may be obtained by methods known to the skilled person in the art (e.g. extraction, fractionation, centrifugation) from various sources (e.g. tissue, body fluids, blood, cell extracts, cell culture, etc.).

Examples of nucleic acid populations are
genomic DNA, e.g. human, mouse, rat etc.
total RNA or subfractions thereof, e.g. tRNA, rRNA, miRNA, mRNA, etc.
herring sperm DNA, cotDNA.

It has been found, surprisingly, that the efficiency of the isolation of target molecules or subpopulations from complex nucleic acid populations can be increased significantly by increasing the complexity of the sample. The addition of further nucleic acid populations increases the “sharpness of separation” of the isolation.

The nucleic acid population mixtures to be analyzed comprise at least two different populations which differ with respect to their source (e.g. species, organism, individual) and/or with respect to their complexity or fragment size. The populations can originate from eukaryotic species, e.g. mammalian species, such as, for example, humans, or prokaryotic species, such as, for example, a bacterium or a viral species, or mixtures of eukaryotic and/or prokaryotic and/or viral species. The various nucleic acid populations can be those of the same species, but also those of different species. The populations can also originate from different organisms of a species, e.g. different human individuals. According to the invention, more than two different populations of nucleic acid molecules can also be analyzed, e.g. 3, 4, 5, 6 or even more populations.

In some embodiments, a nucleic acid population comprises at least 10²¹ different sequences, in other embodiments at least 10¹⁸ different sequences and in some embodiments up to 10¹⁵ different sequences, in other embodiments up to 10¹² different sequences, in other embodiments up to 10⁹ different sequences, in other embodiments up to 10⁶ different sequences, in other embodiments up to 10³ different sequences. The average length of individual sequences of the population can typically be about 20-20,000 nucleotides, e.g. about 100-10,000 nucleotides, for example about 100-600 or about 100-400 nucleotides. In certain embodiments populations of large fragments of typically about 5,000-20,000, e.g. about 8,000-15,000 nucleotides can typically be employed. The nucleic acids of a population can comprise double-stranded or single-stranded DNA, RNA or mixtures thereof.

The nucleic acid populations are preferably non-fragmented or obtainable by fragmentation of chromosomal or extrachromosomal DNA from one or more organisms, e.g. by enzymatic fragmentation, chemical fragmentation, mechanical fragmentation, such as, for example, by ultrasound treatment, or other methods.

The method according to the invention comprises the isolation of target molecules from a sample which contains at least two different nucleic acid populations.

A further improvement in the method is possible by consecutive isolation of target molecules in several successive cycles. In this case, the sample to be analyzed is brought into contact several times in succession with capture molecules, each of which can be identical or different.

In a special embodiment of the present invention the isolation of target nucleic acid molecules is performed in consecutive binding and elution cycles that make use of capture probe matrices of different or the same type. The capture probe matrices can be in all cycles of the same type (e.g. an array) or can be different. For example, the capture probe matrix may be a bead support in a first cycle and an array in the following cycle. Alternatively, a bead may be the capture probe matrix in a first cycle and an in-solution capture library may be employed in the second cycle. The present invention is not limited to these examples, a person skilled in the art will be aware of other useful combinations of capture probe matrices employed for a multi-cycle isolation procedure according to the present invention.

The method according to the invention relates to the isolation of target molecules from two or more nucleic acid populations. The target molecules are conventionally sub-populations of the nucleic acid populations to be analyzed. For example, 10⁵ to 10¹², preferably 10⁵ to 50×10⁶ and more preferably 2×10⁵ to 10⁶ different target molecules can be isolated by the method according to the invention. The number of target molecules to be isolated correlates with the length of the regions of the nucleic acid sequences covered by capture probes. Typical ranges of the nucleic acid sequences which are isolated are 10 kb to 100 Mb, preferably 50 kb to 10 Mb, more preferably 250 kb to 10 Mb, very preferably 500 kb to 4 Mb.

Capture molecules are used for isolation of the target molecules. These are nucleic acid molecules which bind specifically to the target molecules to be isolated, in particular by hybridization in the form of a nucleic acid double strand. The capture molecules are conventionally hybridization probes which are complementary, or at least complementary in part regions, to the target molecules to be isolated. According to the invention, so-called wobble bases (inter alia degenerated bases, abasic sites, universal bases) which are complementary to more than one nucleic acid fragment can also be introduced into the capture probes. The hybridization probes can likewise be nucleic acids, in particular DNA or RNA molecules, but also nucleic acid analogues, such as peptide nucleic acids (PNA), locked nucleic acids (LNA) etc. The hybridization probes preferably have a length corresponding to 10-100 nucleotides and do not have to consist uninterruptedly of units with bases, i.e. they can also contain, for example, abasic units, linkers, spacers etc.

In the method according to the invention, the capture molecules can be immobilized on an array on particles (beads) or on a different solid phase or can be present in the free form, i.e. in solution.

The nucleic acid capture molecules used in the method according to the invention are preferably a population of at least 10, in some embodiments of at least 1,000, in other embodiments of at least 100,000, in other embodiments of at least 10,000,000 different nucleic acid molecules.

Sequences of nucleic acid capture molecules can be derived from databases (e.g. databases in the internet) which contain the nucleic acid sequences of organisms which have already been thoroughly sequenced. Alternatively, the sequences of nucleic acid capture molecules can also be chosen from as yet still unknown sequences, e.g. sequences which are not yet known in the nucleic acid populations to be analyzed.

The capture molecules used in the method according to the invention can be chosen such that they contain sequences of one or more of the nucleic acid molecule populations to be analyzed. In certain embodiments, capture molecules which recognize target molecules from not all of the nucleic acid populations to be analyzed can be chosen, for example capture molecules which recognize only target molecules from one of the nucleic acid population to be analyzed.

In a preferred embodiment of the invention, at least one of the nucleic acid molecule populations, preferably at least one population which contains the target molecules to be isolated, carries a marking. Markings can be detectable groups, for example dyestuffs, fluorescence markings or partners of binding pairs which have bioaffinity, for example haptens, which bind specifically to antibodies, biotin, which binds specifically to avidin or streptavidin, or carbohydrates, which bind specifically to lectins. On the other hand, the marking can also be one or more terminal adaptor nucleic acid sequences which, for example, make amplification possible in subsequent steps.

Several of the nucleic acid populations to be analyzed also can optionally carry markings, wherein individual nucleic acid populations preferably carrying different markings. It is thus possible that in the context of isolation and optionally characterization of the nucleic acid target molecules, these can be assigned to a particular nucleic acid population. The method according to the invention can comprise a single isolation step or several cycles of consecutive isolation and optionally characterization of target molecules. The characterization of the target molecules here preferably comprises a partial or complete sequence determination of the nucleic acid target molecules isolated.

In the context of an isolation procedure consisting of several cycles, an amplification and/or a fragmentation of the target molecule population can be carried out between individual cycles.

In a further embodiment of the present invention, when the nucleic acid populations are brought into contact with the capture molecules, a DNA-binding protein, in particular a DNA-binding protein with a single-stranded DNA-dependent ATPase activity, such as, for example, RecA and optionally ATP, is added.

Preferred embodiments of the present invention are explained in detail in the following:

### Analysis of Host-Pathogen Nucleic Acid Populations

A typical use of the method according to the invention is the analysis of a mixture of nucleic acid populations of a host, in particular of a eukaryotic host, such as, for example, of a mammal, e.g. a human, and one or more pathogens (host-pathogen population mixture). The present invention makes it possible here for the portions of the pathogen to be isolated from the background of the host in a targeted manner and fed to the sequence analysis.

In a first embodiment, the E. coli strain K12 e.g. in a mixture with the pathogenic E. coli strain O157 in the ratio of 1:1,000 (1 ng/1,000 ng) is analyzed for isolation of parts of the nucleic acid population of O157. Probes which are complementary to sequences from E. coli O157 are used as capture probes. The pathogen can be identified by subsequent sequencing.

In a further embodiment, the E. coli strain K12 e.g. in a mixture with human genomic DNA in the ratio of 1:750 (2 ng/1,500 ng) is analyzed for isolation of parts of the nucleic acid population of E. coli K12. Probes which are complementary to sequences from E. coli K12 are used as capture probes. The nucleic acid population isolated can be identified by subsequent sequencing.

In a further embodiment, the pathogenic E. coli strain O157 e.g. in a mixture with human genomic DNA in the ratio of 1:750 (2 ng/1,500 ng) is analyzed for isolation of parts of the pathogenic nucleic acid population of E. coli O157. Probes which are complementary to sequences from E. coli O157 are used as capture probes. The nucleic acid population isolated can be identified by subsequent sequencing.

In a further embodiment, marked and non-marked nucleic acid populations are present side by side in a mixture of the nucleic acid populations to be analyzed. The performance of the isolation can be increased significantly by this means. In the detection of a pathogen in the background of the host, this leads e.g. to an increase in the sensitivity, which is then a decisive advantage in the sequence analysis.

Probes for the pathogen or pathogens to be analyzed are provided as the capture probe matrix. The sample material to be analyzed, which contains nucleic acid populations of the host (e.g. human) and of the pathogen (e.g. E. coli O157) is prepared during the sample preparation in accordance with known protocols of the sequence technology used later and acquires terminal markings (adaptor sequences for later amplification or capturing steps) by this means. A human nucleic acid population of corresponding length which contains no such marking is added to this complex nucleic acid population mixture. As a result of the addition of the non-marked nucleic acid population in the sense of competitive hybridization, the background for the pathogen to be analyzed can be reduced, since the non-marked nucleic acid population indeed participates in the contacting with capture probes, but is not multiplied in the adaptor-based amplification in the following step (since it is without the corresponding marking/adaptor sequences) and is also not detected during the sequence analysis in the following step. According to the invention, the non-marked nucleic acid population (here human genomic DNA) is employed at least in the same amount as the sample material to be analyzed, preferably in a 4- to 10-fold excess, still more preferably in a 10- to 100-fold excess.

Detection of Virus Integration Sites into Host Genomes

Viral integration in host genomes plays an important role for a plurality of pathogenic processes in human or other vertebrates, e.g. mammals, birds, etc. An in-depth-knowledge of the viral integration sites in the host genome bears a huge potential with the mid-term goal of personalized treatment of patients against the viral infection with modern techniques, eg. gene-therapies.

The present invention provides ways for achieving this goal by detecting the respective viral integration sites in the host genome of an infected individual. When screening hundreds or thousands or even larger patient cohorts, the prior-art technology (long-mediated polymerase chain reaction, LM-PCR) comes to its limitation, due to throughput restrictions. The present invention allows for effective detection and screening for viral integration sites by combining isolation/enrichment technology with next generation sequencing technology.

In one embodiment of the present invention, this is achieved by a 3 step process:

### Step 1: Design of the Capture Matrix

Capture probes complementary to one strand or both strands of a target virus are provided on a capture matrix of choice (e.g. biochip, microarray, beads, in-solution baits)

### Step 2: Isolation/Enrichment of Regions of Interest

One or more fragmented nucleic acid population libraries of one or more infected host genome, e.g. a mammalian, particularly human genome, are hybridized with the capture probe matrix of Step 1; after washing away of un-bound fragments, the specifically bound fragments are isolated/eluted. The isolate/eluate contains viral sequences and parts of the host genomes

### Step 3: Sequencing

The eluate/isolate from Step 2 can now be sequenced and the resulting sequencing data can be mapped back to the host genomes to detect the viral insertion sites. This procedure is schematically shown in FIG. 9.

### Use Example

The detection of viral integration into host genomes according to the present invention was used for detecting the integration of the LTR region of foamy virus into the genome of Mus musculus. As negative control, sequences of Lenti virus were represented as capture probes on the capture probe matrix (microarray). After hybridization of the sample to the capture probe matrix, the microarray was washed and the retained fragments of the library were eluted. The eluate was subjected to paired end sequencing (Illumina Genome Analyzer) and an Average Depth of Coverage of over 15.000 was detected. This correlates to the fact that each of the viral LTR bases was called 15.000 times on average. The consensus coverage, hence that each base has been called at least once, was 100%. The 20× consensus coverage, hence that each base has been called at least 20-times, was above 99%. In contrast, the Average Depth of Coverage of the Lenti virus, as a negative control, was 0.

By mapping the paired reads to the viral genome, we found about 1300 read pairs where one read was located in the virus completely, while the second is read was mapped to the mouse genome. Thereby, we were able to detect 22 insertion sites. Of these, 12 have also been detected with LM-PCR while 10 other insertion sites were not detected by this technology.

Furthermore, additional insertion sites can be identified by reads that contain both viral and mouse sequences.

Thus, a further embodiment of the present invention refers to a High-Throughput approach for the detection of viral integration into host genomes.

The high coverage and multiplicity of sequence reads allows for a horizontal and vertical extension of the approach. First, the capacity of the capture probe matrix can be extended to screen for several viruses in parallel (horizontal extension). Furthermore, by employing marked/bar coded libraries of the nucleic acid populations of interest, as many as 100 individuals can be screened in an integrative manner in parallel (vertical extension).

In a special embodiment of the present invention, a capture probe matrix, representing a plurality, e.g. up to 100 different viruses, is contacted with a mixture of a plurality, e.g. up to 100 bar coded nucleic acid populations (e.g. correlating to up to 100 individuals). This allows for a very efficient detection of all combinations of viral insertion sites in all individuals in true High Throughput fashion.

Analysis of Nucleic Acid Populations which Contain Hitherto Unknown Species

A further use of the present invention is the detection of pathogens which are still hitherto unknown from nucleic acid population mixtures. Thus, target molecules from still unknown pathogens can be detected by using as capture molecules those sequences which have a homology to a particular class of pathogens (=common probes).

In a first embodiment, a mixture of various E. coli strains is analyzed. Sequences (common probes) which are common to as many as possible known (and therefore also still unknown strains) are chosen as capture probes. Isolation with subsequent sequencing then provides a breakdown of which E. coli strains were present in the mixture and moreover also information as to whether still as yet unknown strains were represented in the mixture.

In a further embodiment, instead of common probes for a single particular nucleic acid population, common probes for several nucleic acid populations are chosen. By such a procedure it is possible to “fish” for as yet unknown representatives of these particular classes in even considerably more complex nucleic acid populations.

In this context, the human microbiome (entirety of all microbial genomes in a human organism; see HGMI Human Gut Microbiome Initiative; http://genome.wustl.edu/hgm/HGM_frontpage.cgi) can be analyzed.

In the discovery method, “common” capture probes of which the sequence are specific not only for a single but for a class of microorganisms are provided. For each of the classes of microorganisms which are to be fished, common probes are in each case provided. The sample to be analyzed is brought into contact with the capture probes as a complex nucleic acid mixture and the corresponding regions of the classes of microorganisms are isolated in this way. Thereafter, sequence analysis is used to determine which and how many microorganisms were present in the particular sample analyzed. Comparison with sequence or sequencing data of known microorganisms (from databases or internet databases) then makes identification of still as yet unknown microorganisms possible by conclusion. As soon as such a microorganism has been identified, this microorganism or this specific species can be fished for specifically in a subsequent experiment with the corresponding specific capture probes.

By using capture probes which are sequence-specific for a large number of nucleic acid populations, the sequence of which is already known, such a complex mixture can of course be analyzed in a targeted manner. After isolation of the particular sequence sections of interest from the large number of nucleic acid populations, the isolate is then subjected to a sequence analysis.

### Profiling of Complex Nucleic Acid Populations

In a further use, individuals are compared with the aid of their complex nucleic acid populations. Such comparisons make it possible to draw a conclusion on the common features or differences between individuals on the basis of complex nucleic acid populations.

One embodiment example is the comparison of the nucleic acid populations of the human microbiome of various individuals. Specific capture probes for microorganisms, the sequence of which is already known, are used for this. If as many microorganisms as possible, ideally all the microorganisms as yet known for the individuals to be analyzed, of the microbiome are imaged by corresponding capture probes, each individual can be characterized as precisely as possible with respect to the microbiome, or the microbiome fraction represented by capture probes, respectively, and differences or common features can be determined. In this way, tissue-specific signatures for predetermined sequence portions may be effectively compared, wherein conclusions with regard to common features and differences between the analyzed nucleic acid population will be possible.

A further embodiment example is the comparison of the nucleic acid populations of particular tissues of various individuals, e.g. human individuals. The tissues can be e.g. tumors or healthy tissue, tissue of specific origin (brain, pancreas, lung, heart, skin etc.). Specific capture probes for those sequence sections of the human genome for which a detailed analysis is desired are used for this. After the nucleic acid populations have been brought into contact with the capture probes, the desired nucleic acid sequences are bound by the capture probes. After separating off non-bound material, the bound parts of nucleic acid populations can be isolated and fed to the sequence analysis.

### Exon Junction Analysis

The alternative splicing of complex genomes is as yet still understood little. It has as yet been found that most genes are subject to alternative splicing, but nevertheless high throughput methods for investigating this in detail are still lacking.

Analysis of alternative splicing with corresponding microarrays (inter alia Affymetrix, USA) merely allows detection of splice forms which occur very often, and also only those variants which were known at the point in time when the corresponding microarray was produced or designed.

The present invention solves this problem as follows:
provision of RNA, e.g. total RNA, of the samples to be analyzed,
preparation therefrom of a paired-end sequence cDNA library with adaptor sequences, e.g. with the conventional adaptor sequences for an NGS platform (e.g. 454, Illumina, Solid),
designing of specific capture probes, the probes being complementary to the 3′ and 5′ terminal regions of the exons of the genes to be analyzed,
bringing of the capture probes into contact with the paired-end sequence cDNA library,
removal of the fragments not bound specifically to the capture probes,
isolation of the fragments bound to the capture probes,
sequence analysis of the fragments isolated,
mapping of the sequencing results with respect to the exon sequences (all possible combinations of the exons of the particular genes to be analyzed); which exon is joined to which other exons of the particular gene can be determined by this means; this is possible due to the two paired-end sequence reads, which can bridge a defined length (library sizes),
optionally digital counting of the exon junctions.

The capture probes can be employed here on a solid phase or in the liquid phase. A direct comparison between individuals is possible because two and more nucleic acid populations, which can be distinguished by an appropriate marking (e.g. a molecular bar code/index), are simultaneously subjected to the method described above.

Alternatively, one can proceed as follows:
provision of RNA, e.g. total RNA, of the samples to be analyzed,
preparation therefrom of a paired-end sequence cDNA library with adaptor sequences, e.g. with the conventional adaptor sequences for an NGS platform (e.g. 454, Illumina, Solid),
adding of further nucleic acid populations (human genomic DNA or herring sperm DNA or cotDNA or tRNA or mixtures of those nucleic acid populations) to the paired-end sequence cDNA library,
designing of specific capture probes, the probes being complementary to the 3′ and 5′ terminal regions of the exons of the genes to be analyzed,
bringing of the capture probes into contact with the paired-end sequence cDNA library, and the above further nucleic acid populations,
removal of the fragments not bound specifically to the capture probes,
isolation of the fragments bound to the capture probes,
sequence analysis of the fragments isolated,
mapping of the sequencing results with respect to the exon sequences (all possible combinations of the exons of the particular genes to be analyzed); which exon is joined to which other exons of the particular gene can be determined by this means; this is possible due to the two paired-end sequence reads, which can bridge a defined length (library sizes),
optionally digital counting of the exon junctions.

### Analysis of Translocations for Tumor Diagnostics

An essential manifestation of cancer is translocation in cancer-associated genes (http://www.sanger.ac.uk/genetics/CGP/Census/). To be able to demonstrate this, the following procedure is proposed according to the invention:
provision of a nucleic acid population from the genomic DNA to be analyzed,
preparation therefrom of a paired-end sequence library with adaptor sequences, e.g. with the conventional adaptor sequences for an NGS platform (e.g. 454, Illumina, Solid),
designing of specific capture probes; the probes are complementary to terminal ends of the known translocation breaking sites of the genes to be analyzed,
bringing of the capture probes into contact with the paired-end sequence library, and the above further nucleic acid populations,
removal of the fragments not bound specifically,
isolation of the bound fragments,
sequence analysis of the bound fragments,
mapping of the sequencing data with respect to the genomic sequence (with and without a translocation event),
determination and counting of the translocation events for the sample to be analyzed.

The capture probes can be employed here on a solid phase or in the liquid phase. A direct comparison between individuals is possible because two and more nucleic acid populations, e.g. from the genome of a tumor cell and of a normal cell, are simultaneously subjected to the method described above.

Ideally, these analyses are carried out simultaneously by providing the nucleic acid populations of the tumor and the normal state each with a corresponding marking (e.g. molecular bar code/index) which allows assignment to the particular population (tumor or normal) during the subsequent sequence analysis.

Alternatively, one can proceed as follows:
provision of a nucleic acid population from the genomic DNA to be analyzed,
preparation therefrom of a paired-end sequence library with adaptor sequences, e.g. with the conventional adaptor sequences for an NGS platform (e.g. 454, Illumina, Solid),
adding of further nucleic acid populations (human genomic DNA or herring sperm DNA or cotDNA or tRNA or mixtures of the above nucleic acid populations) to the paired-end sequence library,
designing of specific capture probes; the probes are complementary to terminal ends of the known translocation breaking sites of the genes to be analyzed,
bringing of the capture probes into contact with the paired-end sequence library, and the above further nucleic acid populations,
removal of the fragments not bound specifically,
isolation of the bound fragments,
sequence analysis of the bound fragments,
mapping of the sequencing data with respect to the genomic sequence (with and without a translocation event),
determination and counting of the translocation events for the sample to be analyzed.

### Analysis of Variations in the Number of Copies of Genes

In order to detect copy number variations (CNVs) in the context of the CGH method, to date above all microarrays which are built up from long oligonucleotides or BACs have been used. However, this method is limited with respect to sensitivity and robustness.

In order to be able to detect CNV with the highest possible resolution, the following procedure is proposed according to the invention:
provision of a nucleic acid population of the genomic DNA to be analyzed,
preparation therefrom of a sequence library with adaptor sequences, e.g. with the conventional adaptor sequences for the NGS platform (e.g. 454, Illumina, Solid),
designing of specific capture probes; the probes are complementary to regions in the genome which are to be analyzed for CNV,
bringing of the capture probes into contact with the sequence library,
removal of the fragments not bound specifically,
isolation of the bound fragments,
sequence analysis of the bound fragments,
mapping of the sequencing results with respect to the genomic sequence and
counting of the copies for the sample to be analyzed.

If instead of a genomic population to be analyzed a mixture, of indexed/marked populations (e.g. provided with molecular bar codes; after sequencing the pool and therefore the underlying sequence information can then be decoded), copy number variations can be deduced directly from the data of the NGS sequencing.

Alternatively, one can proceed as follows:
provision of a nucleic acid population of the genomic DNA to be analyzed,
preparation therefrom of a sequence library with adaptor sequences, e.g. with the conventional adaptor sequences for the NGS platform (e.g. 454, Illumina, Solid),
adding of further nucleic acid populations (human genomic DNA or herring sperm DNA or cotDNA or tRNA or mixtures of the above nucleic acid populations) to the sequence library,
designing of specific capture probes; the probes are complementary to regions in the genome which are to be analyzed for CNV,
bringing of the capture probes into contact with the sequence library, and the further nucleic acid populations,
removal of the fragments not bound specifically,
isolation of the bound fragments,
sequence analysis of the bound fragments,
mapping of the sequencing results with respect to the genomic sequence and
counting of the copies for the sample to be analyzed.

### Multiplexing

To analyze as many nucleic acid populations as possible in parallel, so-called multiplexing is appropriate. In this, each nucleic acid population is marked by a so-called code (or bar code, index or molecular bar code). After sequence analysis of the mixture of several nucleic acid populations together, due to the coding of the individual populations it is possible to assign the sequence data obtained to the particular populations.

Codes (bar codes, indices) which are introduced during sample preparation of the particular nucleic acid populations are known from the literature. This is effected, inter alia, by introduction of the bar codes in the context of primer sequences by PCR steps.

A further possibility of performing multiplexing results from physical separation of the particular nucleic acid population sections to be analyzed.

Further methods and applications of markings/bar codes/indices are described in DE 10 2008 061 774.1 and U.S. 61/121,615. The contents of these documents are herein incorporated by reference.

### Use Example

In the context of process optimization, various process parameters are to analyzed by the multiplex method for development of a cancer chip. 112 cancer genes are to be analyzed per sequence analysis. In order to determine the optimum experimental conditions for selection of the cancer genes from the complex nucleic acid population (human genomic DNA), capture probes specific for 8×14 different cancer genes and 8 patient samples are provided. In each case 14 cancer genes represent an experiment unit. These are provided physically separated (e.g. 8 individual arrays, 8 individual bead libraries, 8 individual capture probe libraries in solution). 8 experiments are carried out, 8 different process parameters (inter alia buffer conditions, elution conditions, temperature conditions, probe length etc.) being used. After the samples have been brought into contact with the corresponding capture probes, the non-bound parts of the particular nucleic acid populations (samples) are removed and the bound parts are isolated. After isolation of the bonded parts of the nucleic acid populations of the 8 separate experiments, the 8 samples are combined again and evaluated via a sequence analysis. By correlation of the sequence data to the particular experiment units (and therefore the particular process parameters used), an optimized set of process parameters can be determined very effectively and rapidly by the multiplex method.

### Consecutive Multiple Isolation

A further possibility, the performance of the isolation of nucleic acid sequences from two or more complex nucleic acid populations comprises bringing them into contact with capture probes two or several times. In this procedure, for one isolation step a first set of capture probes is used for bringing into contact with the nucleic acid population, for a second isolation step a second set, and optionally for further isolation steps further sets of capture probes. According to the invention, the sample is first brought into contact with the first set of capture probes, the non-bound constituents of the nucleic acid populations are removed and the bound constituents are isolated. In order to make the nucleic acids isolated available for a further isolation step, it may be appropriate first to amplify the nucleic acids isolated in order to provide sufficient material. The nucleic acids isolated in the first step are then—where appropriate after amplification—brought into contact with the second set of capture probes. The non-bonded constituents are removed and the nucleic acids bound are isolated. If an even higher performance is required, further isolation steps can be carried out, before the isolate is then subjected to a sequence analysis.

According to the invention, the first, the second and further sets of capture probes can be identical. It may moreover be necessary for the first, second and further sets of capture probes to be different. Mixed forms of identical and different sets of capture probes are equally possible.

The performance of the isolation after the first, second and further isolation cycles can furthermore be monitored by sequence analysis. According to the invention, as many isolation cycles to achieve the required performance can be carried out.

One criterion which is essential for the performance, namely the homogeneity of the isolation, can be increased very effectively according to the invention via consecutive multiple isolation. While in a first cycle of the isolation of nucleic acid sequences from nucleic acid populations particular target sequences are still under-represented and therefore possibly fall below the detection limit of the sequencing apparatus, these can be made available in a higher number of copies by second (or correspondingly further) isolation cycles following after the amplification. That is to say these regions which could not be analyzed or not detected previously can now be analyzed via the sequencing apparatus after one or more further cycles. The method according to the invention is thus a method for increasing the sensitivity of the sequencing technology.

Regions which were very different with respect to their representation in a first isolation cycle can furthermore be homogenized efficiently with respect to their representation by a second (or further) isolation cycle. The method according to the invention is therefore a method for homogenizing the representation of nucleic acid fragments.

In a special embodiment of the invention a first and the consecutive isolation steps can be performed within the same identical capture probe matrix. Hereby, the capture probes are brought into contact with the nucleic acid population and unbound material is washed away. Afterwards, the targets are released (dehybridized) from the capture probes (e.g. by denaturation, heating). After release (dehybridization) of the targets another binding cycle is carried out within the very same capture probe matrix and again unbound material is washed away. This procedure may be repeated for several times before the enriched targets of interest are eluated/isolated.

### Use Examples:

Consecutive isolation of human genes (BRCA1, BRCA2, TP53, KRAS) from a complex mixture of nucleic acid populations with different capture probe sets.

The complex mixture of 3 nucleic acid populations is composed of human genomic DNA, human tRNA and herring sperm DNA. The capture probes for isolation of the human genes BRCA1, BRCA2, TP53 and KRAS, which comprise the highly complex regions (high-complexity regions) of the human genome, are generated from a database (NCBI: hg 18). Two sets (set A, set B) of capture probes are generated for each of the genes BRCA1, BRCA2, TP53 and KRAS to be isolated. The capture probes of set A and B differ here. The mixture of 3 nucleic acid populations to be analyzed consisting of human genomic DNA, human tRNA and herring sperm DNA is brought into contact with capture probe set A, the non-bonded constituents are removed, and the bonded constituents are subsequently isolated. Thereafter, the nucleic acids isolated are amplified with the aid of a PCR or another amplification technique known to the skilled person and brought into contact with the capture probe set B. The non-bonded constituents are removed and the bonded constituents are subsequently isolated. After two rounds of isolation, the nucleic acids isolated are subjected to a sequence analysis. The capture probe sets A or B may be present on an array or on particles (beads) or immobilized on another type of solid phase or be present in free form, i.e. in solution.

Consecutive isolation of human genes (BRCA1, BRCA2, TP53, KRAS) from a complex mixture of nucleic acid populations with identical capture probe sets.

The complex mixture of 3 nucleic acid populations is composed of human genomic DNA, human tRNA and herring sperm DNA. The capture probes for isolation of the human genes BRCA1, BRCA2, TP53 and KRAS, which comprise the highly complex regions (high-complexity regions) of the human genome, are generated from a database (NCBI: hg 18). Two sets (set A, set B) of capture probes are generated for each of the genes BRCA1, BRCA2, TP53 and KRAS to be isolated. The capture probes of set A and B are identical here. The mixture of nucleic acid populations to be analyzed consisting of human genomic DNA, human tRNA and herring sperm DNA is brought into contact with capture probe set A, the non-bonded constituents are removed, and the bonded constituents are subsequently isolated. Thereafter, the nucleic acids isolated are amplified with the aid of a PCR and brought into contact with the capture probe set B. The non-bonded constituents are removed and the bonded constituents are subsequently isolated. After two rounds of isolation, the nucleic acids isolated are subjected to a sequence analysis. The capture probe sets A or B may be present on an array or on particles (beads) or immobilized on another type of solid phase or be present in free form, i.e. in solution.

### Increasing Performance by RecA

The use of RecA, e.g. heat-stable RecA, obtainable from www.biohelix.com, for bringing a complex mixture of nucleic acid populations into contact with the capture probes makes it possible to increase performance. RecA, as a DNA-binding protein with an ssDNA-dependent ATPase activity, initially bonds to the single-stranded capture probes and actively assists specific bonding to the target molecules.

### Use Example:

Bringing the capture probes into contact with RecA in RecA buffer. Addition of ATP to the mixture of the nucleic acid populations. Subsequent addition of the mixture of nucleic acid populations to which ATP has been added to the RecA/capture probes mixture. Incubation. RecA assists specific bonding to the capture probes. Removal of the parts of the nucleic acid populations not bonded to the capture probes. Isolation of the bonded parts of the of the nucleic acid populations. Sequence analysis of the isolate.

Isolation of Nucleic Acid Populations for Sequence Analysis with the Roche 454 Sequencing Technology

For successful sequencing by means of a Roche/454 sequencer, a DNA sample must be fragmented and modified. In particular, it is necessary to ligate two different adaptors on to the DNA fragment ends and to immobilize these molecules obtained in this way individually on individual beads. These are then amplified in an emulsion PCR, which leads to clonal beads which carry a large number of copies of the same DNA fragment and can be used for the sequencing. In the protocols known to the person skilled in the art for generating DNA libraries (see e.g.: GS DNA Library Preparation Kit Quick Guide, GS 20 Training Guide Version II, GS emPCR Kit Quick Guide, GS emPCR Kit User's Manual, GS FLX DNA Library Preparation Kit User's Manual, GS FLX Sequencing Method Manual), there is the possibility of carrying out an enrichment of desired sequences at various steps.

The following steps are carried out for generating a library in the protocols known to the person skilled in the art:

1. DNA fragmentation (nebulization) or LMW DNA quality determination
2. Fragment end polishing
3. Adaptor ligation
4. Library immobilization
5. Filling reaction
6. Single-stranded template DNA (sstDNA) library isolation
7. sstDNA library quality determination and quantification.

Sequence-specific enrichments can be carried out after, before or during one, several or all of these steps. A particularly preferred step for carrying out a sequence enrichment is step 6. In this, single-stranded DNA fragments are obtained selectively with two different adaptors A and B from a mixture of double-stranded fragments with randomly distributed adaptors (AA, AB, BB). One of the adaptors is biotinylated on one strand, and the fragments are bonded to streptavidin-presenting beads. Fragments which contain only adaptor without biotin are removed by a non-denaturing washing step. In a subsequent denaturing washing step, single-stranded fragments which contain no biotin are eluted selectively from the beads. The biotin-containing counter-strand remains bonded, as do fragments which carry two biotin-containing adaptors.

In a particularly preferred embodiment, desired sequences are enriched, as described, from the fragments obtained in this way. The sample is optionally multiplied beforehand by an LMA (linker mediated amplification) known to the person skilled in the art, preferably using the two adaptor sequences as primer bonding sites, it being possible for one of the two primers to be biotinylated. After an enrichment, the sample can optionally be amplified again and subjected to protocol step 6 again, as described, as a result of which a single-stranded library with two different adaptors is again obtained.

The following protocol sequence thus results:
gDNA fragmentation (200-300 bp, 3-5 μg)
removal of small fragments (beads)
adaptor ligation (polishing)
sstDNA library production (beads)
(optional: pre-enrichment adaptor PCR)
HybSelect (sequence-specific enrichment according to the present invention)
adaptor PCR after enrichment
library capture+emPCR (beads)
library bead enrichment
sequencing primer annealing
next generation sequencing

### Use of Long Nucleic Acid Sections

For enrichment of defined nucleic acid sections, methods are known from the literature which fragments the nucleic acid population to be analyzed into short (ABI-Solid: <100 bp, Illumina-Genome Analyzer<400 bp, Roche-45<500 bp) nucleic acid sections (by ultrasound or nebulizer). At short reading distances of the sequencing apparatus above all this has the decisive disadvantage for isolation of the relevant nucleic acid regions that the capacity of the capture probe matrix (on a solid phase or in solution) is poorly utilized.

According to the invention, the nucleic acid populations are split into the largest possible fragments of e.g. 5-20 kb, the isolation of the nucleic acid regions is carried out with these large fragments and the large fragments are subsequently brought into the sizes of e.g. 90-500 bp required for the particular sequencing technology. This has the decisive advantage that the capacity of the capture probe matrix is utilized considerably better, i.e. more information/data can be isolated with the identical capture probe matrix.

### Use Example:

The nucleic acid populations to be analyzed are broken down into fragments approx. 10 kb in size. Isolation of the nucleic acid regions according to the present invention is carried out with these populations. After isolation, the nucleic acid target molecules isolated are subjected to a fragmentation, from which a fragment size of approx. 400 bp results. In a subsequent step the nucleic acid population is provided with appropriate terminal adaptor sequences, e.g. suitable for the Illumina Genome Analyzer (see Library-Kit Illumina Genome Analyzer). A sequence analysis is then carried out.

In a particular embodiment, several isolation cycles are carried out with different fragment sizes of the nucleic acid populations.

### Use Example:

The nucleic acid populations to be analyzed (e.g. mixture of human genomic DNA and tRNA) are broken down into fragments 2-5 kb in size. The isolation of the nucleic acid regions is carried out with these populations. After isolation, the nucleic acid populations isolated is subjected to a fragmentation, from which a fragment size of 400 bp results. In a subsequent step the nucleic acid population is provided with appropriate terminal adaptor sequences, e.g. suitable for the Illumina Genome Analyzer (see Library-Kit Illumina Genome Analyzer). An amplification via a PCR is carried out on the basis of the adaptor sequencer, in order to make sufficient material available for a further isolation cycle. This isolation cycle is now carried out with a fragment size of 400 bp. After isolation of the nucleic acid sequences of interest and a PCR with 15 cycles based on the adaptor sequences, a sequence analysis is carried out.

### Multi-Cycle Isolation Employing Different Capture Probe Matrices

The nucleic acid populations to be analyzed are contacted in a first step with a bead-based capture probe matrix. In a second and in a third step they are contacted with array-based capture probe matrices.

The nucleic acid populations to be analyzed are of human origin. The regions of interest are the high-complexity regions of the cancer-related genes BRCA1, BRCA2, KRAS and TP53. In the first step the capture probe matrix is a bead-based matrix with capture probes generated from immobilisation of a cotDNA nucleic acid population onto magnetic beads. The nucleic acid populations in form of a DNA fragment library (sequencing library) to be analyzed are contacted with the bead-based capture probe matrix for hybridisation to occur, the unbound material is separated from the material bound to the beads. For the second step the unbound material from step 1 is mixed with additional nucleic acid populations (tRNA and/or herring sperm DNA) and contacted with the second capture probe matrix, which is an array containing probes that were designed to bind the high-complexitiy regions of BRCA1, BRCA2, KRAS and TP53. After hybridisation the unbound material is washed away. The bound material is eluted from the array, subjected to an amplification step (PCR with primers corresponding to the terminal sequencing adaptors of the fragment library). Afterwards, in the third step the amplified material from step 2 is subjected to hybridisation to an array-based capture probe matrix designed to bind the high-complexitiy regions of BRCA1, BRCA2, KRAS and TP53. After hybridisation the unbound material is washed away. The bound material is eluted from the array, optionally subjected to an amplification step (PCR with primers corresponding to the terminal sequencing adaptors of the fragment library) and analyzed on a next generation sequencing platform.

The bead-based capture probe matrix of step 1 is generated by biotinylation of cotDNA (e.g. 3′-biotinylation by use of biotin-16-UTP and terminal transferase) and immobilisation of the biotinylated cotDNA to streptavidin-coated magnetic beads. Alternatively the biotinylated cotDNA may be immobilized to Streptavidin-agarose or -sepharose in a column in order to obtain an easy to use “flow-trough” capture probe matrix. Other ways of immobilizing biotinylated nucleic acid fragments to solid supports are also suitable.

Alternatively other ways of labelling the nucleic acid population may be employed. Furthermore more then one labelled nucleic acid population (combinations of cotDNA, tRNA, herring sperm DNA, etc.) may be immobilized to a solid surface.

In a special embodiment the nucleic acid population that is contacted with the first capture probe matrix is either a unfragmented or a fragmented sequence library that carries terminal sequencing adaptors.

### Concatenation

For next generation sequencing routinely the nucleic acid population of interest is fragmented by mechanical, chemical or enzymatical manipulations in order to produce a fragment library. This fragment library has preferably a size distribution of 100-800 bp. This size distribution is suitable for hybridisation-based isolation/enrichment purposes and is in line with the requirements for next generation sequencing instruments with read lengths of 25-150 bp (e.g. Illumina Genome Analyzer, ABI Solid) or up 500 bp (Roche 454 GS FLX).

For applying hybridisation-based isolation/enrichment technologies of the present invention to third-generation sequencing technologies (e.g. Pacific Biosystems, nanopore sequencing), that are capable of longer read lengths (>500 bp), the fragments of the nucleic acid library may be concatenated after the hybridisation-based isolation/enrichment step before being subjected to next sequencing technologies (third generation or higher) capable of longer sequencing reads. The concatenation process may use enzymatic or chemical ways for joining the fragments of the isolated/enriched nucleic acid library. By following this procedure the increased read length capabilities of the third generation sequencing technologies is efficiently utilized.

### EXAMPLE

### Random Concatenation

The isolated/enriched library is heated up to 95° C. for 3 min and afterwards quickly cooled down to 0° C. by means of an ice bath in order to prevent perfect re-hybridisation (perfect duplex-formation) of the complementary strands. Therefore, a random hybridisation is achieved, resulting in gaps between hybridized fragments. By use of DNA-Polymerase I of Escherichia coli. the gaps can be closed and longer fragments are obtained.

### Example

### Directed Concatenation/Splint-Ligation

In a first step the isolated/enriched library is phosphorylated at the 5′-end by use of ATP and T4 polynucleotide kinase (PNK) and purified to remove the reagents. Next the phosphorylated isolated/enriched library is combined with an excess of adaptor-oligonucleotides (splints) that are partially complementary to both the 3′- and the 5′-sequencing adaptor sequences of the corresponding sequencing technology. These adaptor oligonucleotides function as a splint for a template-directed ligation reaction to join short isolated/enriched fragments of the sequencing library to form longer nucleic acid stretches to be sequenced by techniques capable of longer read lengths (>500 bp). After heating the isolated/enriched library together with the adaptor oligonucleotides to 95° C. for 3 min, the mixture is slowly cooled down to room temperature. Then T4 DNA ligase is added and the template-directed ligation is carried out at 37° C. Afterwards the formed concatenated fragments are purified from the reagents.

Alternate ways of generating longer fragments from the shorter isolated/enriched libraries include assembly-PCR procedures known from gene synthesis protocols or LCR procedures.

By applying hybridisation-based isolation/enrichment technologies by means of concatenation to third-generation sequencing technologies capable of longer read lengths after the present invention, the labelling (bar code/index) of the input nucleic acid population is maintained. Concatenation results in the presence of more label moieties (bar code/index) in long fragments, which can be easily split into the initial short fragments and correlated to the individual nucleic acid populations (e.g. individuals) by bioinformatics (e.g. by making use of adaptor sequences).

### Single Molecule Techniques

The teaching of present invention is not limited to isolation/enrichment of nucleic acid populations for subsequent use by analysis technologies that rely on the detection of a plurality of individual molecules. The person skilled in the art will recognize that the isolated/enriched nucleic acid populations are also well suited for use with single-molecule technologies.

### Recursive Walking

The standard method to analyze sequencing data generated by capturing clones via anti-sense hybridization is to map the sequencing reads back to the original reference sequence used to design the capture probes. As the sequencing reads are relatively short a rather stringent set of alignment criteria is utilized to assure proper alignment between the reads and the reference in order to eliminate false positives. As an example of the mapping criteria used, in cases of reads of length 32 bp, 30 bases over the length of the read are expected to map perfectly with the reference (allowing for 2 mismatches) or they are considered off-target. Serious limitations to this method include, but are not limited to the following:
1. During the process of pre-filtering the raw sequencing reads for quality, it is typical that the reads be compared against the entire reference genome sequence from which they are derived. Natural variations in the form of deletions in the reference sequence will result in sequence reads being ‘flagged’ as foreign to the host genome, and thus eliminated as off-genome reads. FIG. 10 (Next generation sequencing: Comparison to Reference) outlines how sample one has an insertion with respect to the reference, while sample 2 has a deletion with respect to the reference.
2. Inserts, and in particular deletions, in the reference sequence will result in problematic alignments at these junctions between the reference and the reads. FIG. 11 (Next generation sequencing: dealing with insertions) illustrates how this phenomena disqualifies sequencing reads from being considered valid, on-target reads. In this case there is an insertion in the sample being sequence relative to the reference. Reads that span this region are considered off-target and discarded.
3. In cases of genomes that have not yet been fully sequenced there is no complete reference to utilize for the mapping process. The example illustrated in FIG. 12 (Recursive Walking: “Walking” into flanking regions) from the tomato genome is illustrative of this.

The approach being described uses an iterative methodology to cleanly identify and assemble on-target genome reads that overlap with natural breaks in the reference genome as compared to the genome being sequenced. The process begins with the typical assembly of the sequenced reads being mapped to the reference genome. Due to the nature of the mapping process locations of indels between the sample and reference will result in a regions of weak coverage in the sample assembly. This newly assembled consensus sequence is broken at these weak junctions and each of these sub-fragments is used in the iterative process called ‘recursive walking’ and is illustrated in FIG. 13. (Next generation sequencing: Recursive walking). Recursive walking starts with the seed sequence being compared to ALL of the reads from the sequencing run. A more lenient set of criteria are utilized when mapping this seed sequence to the raw sequencing reads, but as an example an overlap of at least 20 bases with perfect identity is a typical, but not exclusive, criteria utilized. Reads that meet these criteria are gathered and assembled together with the seed sequence to form a new consensus sequence that is now longer than the seed sequence for the given round. This process is continued using this new and extended seed sequence until no new reads are identified, and as illustrated in FIG. 13. (Next Generation Sequencing: Recursive Walking)

FIG. 12 (Recursive Walking: “Walking” into flanking regions) shows an actual example from the Tomato genome. The tomato genome to date has not yet been fully sequenced, and the use of the enrichment/isolation technology of the present invention is to identify novel sequence information. In this particular case a reference sequence of length 241 bases was used to design capture probes for enrichment/isolation of the genomic region of interest. Through the “Recursive walking” strategy it was possible to extend this region to 474 bases in four iterations. The colored regions each represent new sequence stretches added to the assembly at each iteration, therefore extending into the previously unknown region. The fifth iteration returned no new raw sequencing reads, and the process for this seed comes to an end.

This recursive process is carried out for each seed sequence and independently extended as far as possible. Since the seed sequences are extended using the Next Generation Sequencing data from the sample, and not being biased by the reference sequence, inserts and deletions (relative to the reference) are naturally assembled into the new consensus sequence in a de novo fashion. The resulting extended seeds are then assembled together to form a final consensus sequence that bares new information as compared to the reference.

Selecting Capture Probes with Improved Capturing Performance

Independent from the selected capture probe matrix (e.g. array, beads, in-solution baits, . . . ) it is of high importance that the capture probe, is capable of binding the target of interest with high specificity. This includes that the capture probe only binds to the target of interest, but also that a plurality of capture probes exhibit similar or ideally the same capture performance. If the latter is not the case, the targets of interest out of the nucleic acid populations will be enriched/isolated with different performance levels. This will hamper the subsequent sequence analysis dramatically since more or less the target of interest with the least capture performance will determine the overall performance of the assay. This translates for the subsequent sequence analysis to an increased need of sequencing, adding additional cost to the analysis.

Various studies performed by the inventors revealed that it is not a priori predictable by calculations that a certain capture probe will have a specific binding performance. or a plurality of different capture probes will have comparable or the same capture performance. This results in a need for methods to improve capture probe performance on the one hand or on the other procedures that allow the selection of capture probe with higher capture performance from a large pool of capture probe of unknown capture performance on the other hand.

The present invention provides procedure and methods for selection of better or optimal capture probes from a plurality of capture probes with unknown capture probe performance.

In conventional capturing assays the relationship between the capture probe and the assay result is linear, therefore directly related. Therefore it is easy to correlate the capture probe performance to an individual capture probe or compare individual capture probe performances among each other.

In contrast, this is not the case when the nucleic acid population library is employed which is ruled by a poison distribution. Therefore, the result—hence the sequence data point (sequence tag, or sequence read) is not directly related to an individual capture probe of the capture probe matrix. This is due to the fact that one capture probe is capable of capturing a plurality of different fragments of the nucleic acid population library. This even gets worse when several capture probes, that are situated in close sequence proximity, are used that all have a certain likelihood of capturing the same library fragments.

The present invention provides methods to correlate the sequencing result (sequencing data point, sequencing read) directly to the capture probe that is responsible for capturing individual library fragments. And furthermore, the present invention provides methods for correlating the capture probe performance of individual capture probes and additionally methods for subsequent selection of optimal capture probes or capture probes with increased capturing performance.

When several capture probes are designed for capturing a certain target and these probes are situated within close spatial proximity in respect to the target, it is not possible to compare the performance of the individual capture probes or directly relate the sequencing data to the individual capture probe. To resolve that problem according to the present invention, the capture probes that are in close proximity are physically separated between several capture probe matrices. Next the nucleic acid populations (fragment libraries) are contacted with these separated capture probe matrices individually (e.g. when 16 matrices are used, accordingly 16 aliquots of the nucleic acid population/fragment library have to be employed). The number of different capture probe matrices that are required to maintain the direct correlation between capture probe and sequencing results is dependent on the proximity/distance between the capture probes and the fragment library size (the size distribution of the fragment library).

When the fragment library has a distribution from 100 to 150 bp, with 95% of its members being within that interval, the maximum fragment size F is 150 bp. When then the capture probes (probelength L is 50 bp), designed for being in close spatial proximity to each other, have a distance D of 8 bp, the number of different capture probe matrices required is N=(L+(F−L))/D=(50+2*(150−50)/8=31. This number is guarantees a direct relationship between capture probe and sequencing result since the next capture probe represented on the individual capture probe matrix is spaced so far away that it is not capable of hybridizing to the same library fragment. After the nucleic acid population have been hybridized to the separate capture probe matrices and the unbound material was washed away, the retained fragments are eluted/isolated. Afterwards the eluates are subjected to sequencing analysis. This can be done by sequencing all eluates separately. Alternatively, in a special embodiment of the invention the fragment libraries that are to be employed are marked (indexed with a bar code) before being hybridized with the individual capture matrices. Therefore, each capture matrix is hybridized with a samples that has a different bar code, resulting in a plurality of bar coded eluates. The bar code eluates can be combined into a pool/mixture and can be sequenced together. This reduces cost for sequencing while the direct relationship between capture probe and sequencing results is maintained by use of the bar code, although the eluates are sequenced as a mixture. This makes this a very effective way of comparing capture performance between capture probes and selecting the best or comparable performers.

In a special embodiment of the present invention the performance of the capture probes is laid down and collected in a database. This flexible and continuously growing data repository allows to select the optimal probes for a broad spectrum of applications, such as:
SNP-Typing: select the best probe or probes for capturing targets that contain SNPs
Mutation-Screening: select the best probe or probes for capturing targets that contain a mutations
Exon-Sequencing: select the best probe or probes for capturing exonic regions
miRNA-Sequencing: select the best probe or probes for capturing regions that contain miRNA-genes
Copy Number Variation: select the best probe or probes that allow for detection of copy number variation with the least bias
SNP-Typing: select the best probe for capturing targets that contain SNPs with a frequency>0.5

This “Good Probe Database” allows for a flexible design of a plurality of custom capture probe matrices (e.g. microarrays, beads, in-solution baits, membranes, microtiter plates). These custom capture probe matrices can be employed either for isolation of nucleic acid populations as described above or even for conventional analytical applications. e.g. SNP-typing arrays, mmRNA-arrays,

### Example

### Identification of Oligonucleotide Probes with the Best Capture Performance for the Design of an Optimized Cancer Exome Biochip

This example translates to the question: “find the best 25 (or 50) probes per kilobase of target region (translates to 5 (10) probes per exon). This approach may be used to form various products, e.g. a Cancer-Exome Standard biochip (with 25 probes per kilobase/5 probes per exon=selection of the 5 probes with the best capture performance) or a Cancer-Exome Deep biochip (with 50 probes per kilobase/10 probes per exon)=selection of the probes with the best capture performance)

For identification of capture probes it may be ideal to combine 2 approaches/technologies:

(a) Fluorescence-based microarray hybridisation; strength: assessing individually a large number of probes in a small number of genes (regions of interest)
(b) Nextgen sequencing; strength: assessing individually a small number of probes in a large number of genes (regions of interest)

This combined approach is especially helpful, if in a first phase (microarray) the probes are screened at a very deep tiling-scheme. Otherwise it may be better to just straightforward start with the NGS phase

The workflow would contain 2 phases:

Phase 1: microarray

### Array-Design/Tiling

| ROI | Size, kb | tiling | 1 bp | 5 bp | 10 bp |
|---|---|---|---|---|---|
| cancer genes | 500 | probes | 1000000 | 200000 | 100000 |
| 115 genes | | probes/kb | 2000 | 400 | 200 |
| 2100 exons | | probes/exon | 400 | 80 | 40 |

taking into account: ss and as strands, exon size = 200 bp

To screen at a 1 bp tiling, a lot of probes/array are required. It would be desirable to get a larger size of a target region covered within one array. Furthermore, at a 1 bp tiling, the sequence homology (“similarity”) of 2 subsequent probes (at 50 bp length) would be 98%. Employing e.g. a 10 bp tiling scheme the sequence homology of 2 subsequent probes is 80%, which is reasonable. An alternating tiling scheme of 50 mers on sense and antisense strand should be implemented. From hybridisation of PCR-products it is well known that both strands behave quite different. A 10 bp alternating tiling scheme translates to 200 probes per kilobase or 40 probes per exon. The tiling represents the first (random) filter of capture probe selection. One may have to implement some additional criteria for the tiling in order to make sure that: each small part of a region of interest (e.g.) exon is covered with sufficient probes and some probes will have to be ruled out due to high sequence homology within the genome (use repeat masking oder frequency of 15 mers).

Performing the microarray hybridisation experiment is the second filter. For classifying better from poor performing capture probes, the fluorescence intensity upon hybridisation with a labeled sequencing library is employed The goal is to reduce the 200 probes/kb (40 probes/exon) to a target value of 88 probes/kb (21 probes/exon). Therefore, the intensities of the probes are ranked and the best 21 probes are further processed in Phase 2 (NGS). In addition it has to be taken into account that small targets (e.g. exons) are covered with enough probes (=additional criteria for ranking)

### Phase 2: NGS

In this phase NGS & multiplexing with 16 bar codes is implemented in order to establish a clear 1:1 link between a sequence-tag and the capture probe on the microarray that did capture this sequence. Therefore 16 arrays are implemented.

Probes that are close to each other (closer than twice the library size) are placed not into the same array. Probes that have a greater distance than twice the library size can be put into the same array. Each of the 16 arrays is hybridized with a sequence library having an individual bar code (altogether 16 bar codes). Therefore, a 1:1 relation between sequence tag and probe is maintained. The sequencing results are deconvoluted on the basis of the coverage data and the relationship between bar code and capture probe. From this again a ranking of capture probes is established. The performance (ranking and additional criteria) of probes is stored into a database. On the basis that 80 probes/kb are screened within Phase 2, 1 NGS run will be able to screen ˜3100 exons (˜620 kb) starting from 16*15624=249.984 probes to select the best probes for sequence capture. Result is an optimized Cancer Exome design within 1 array.

### FIGURES

FIG. 1:

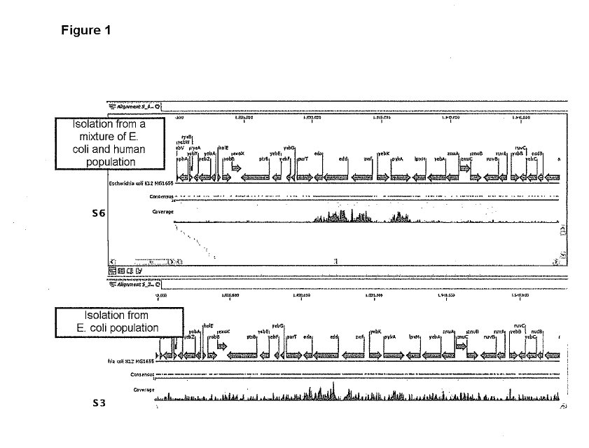

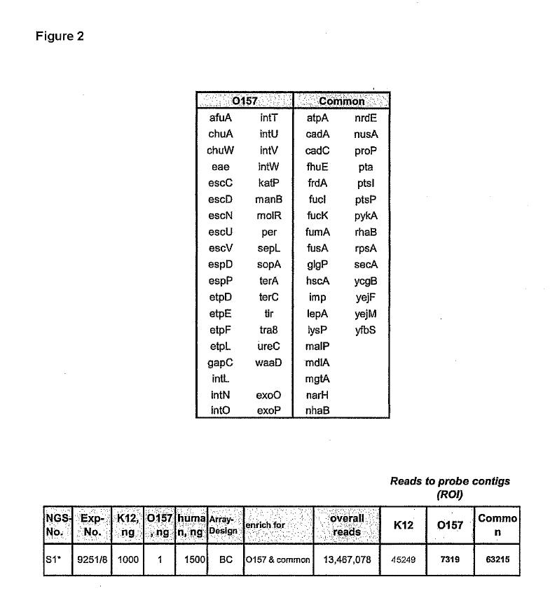

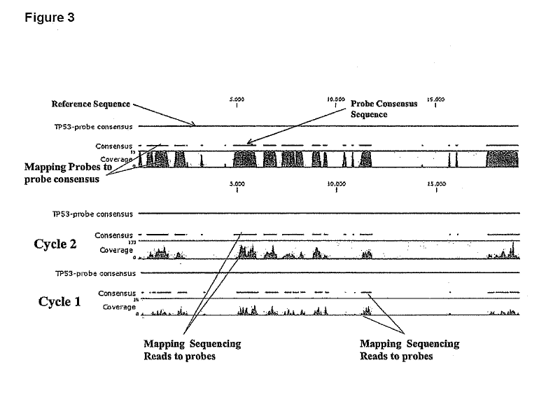

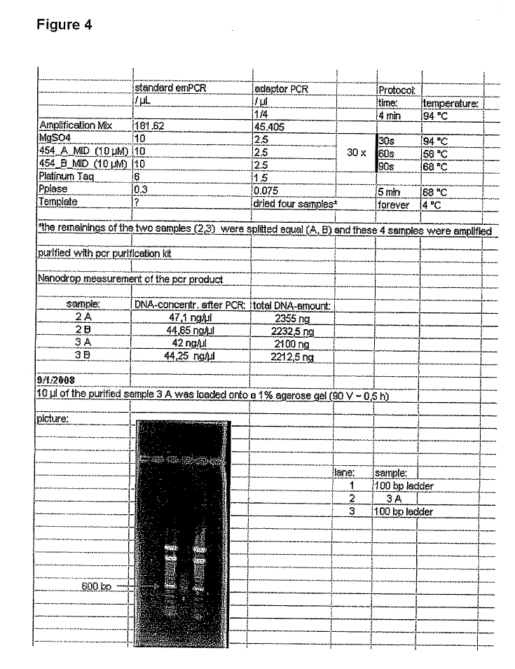

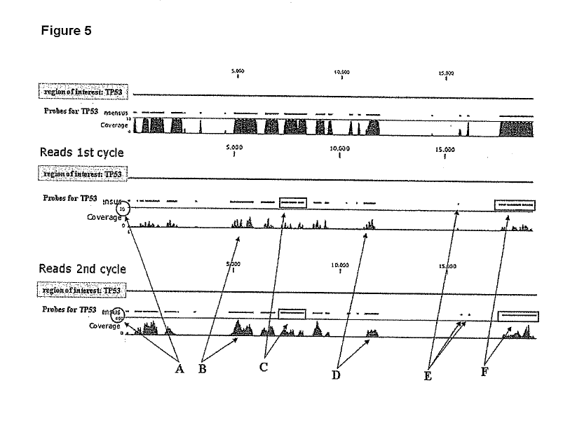

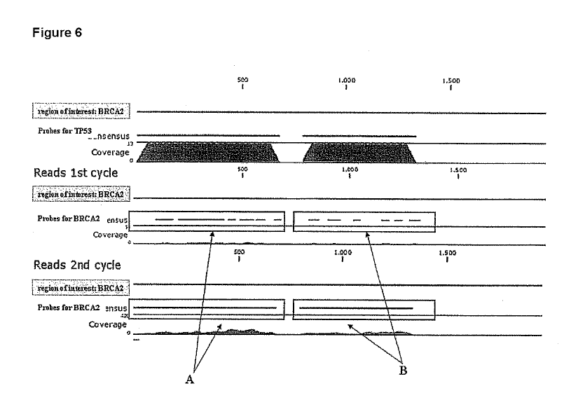

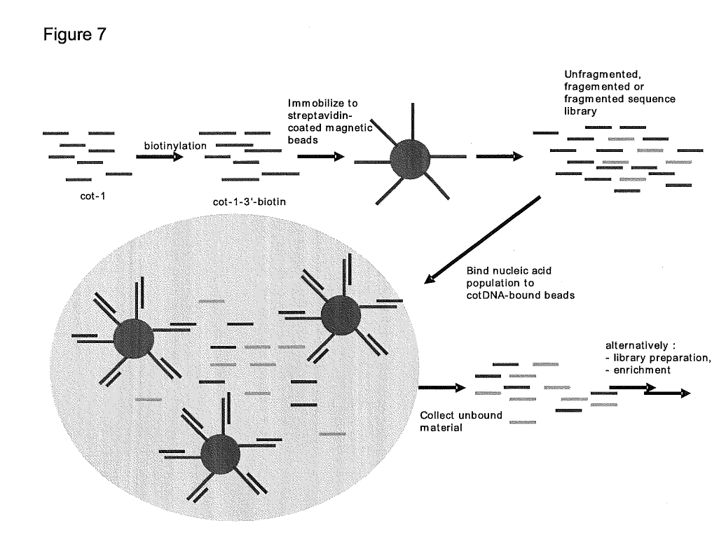

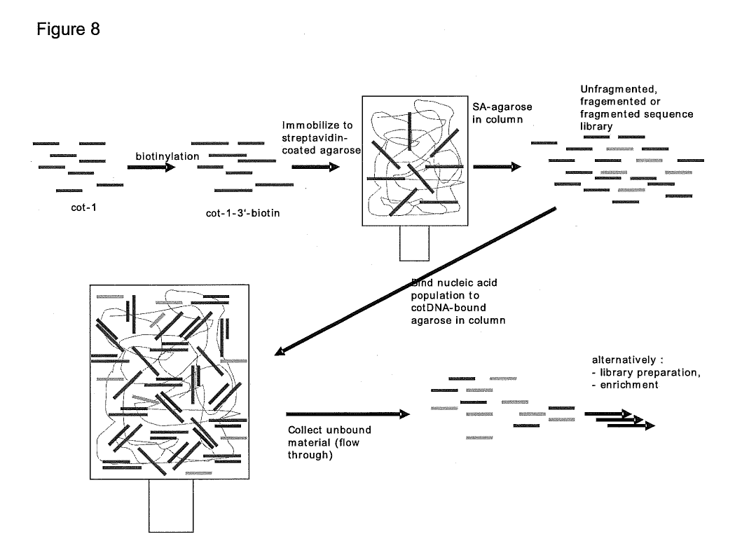

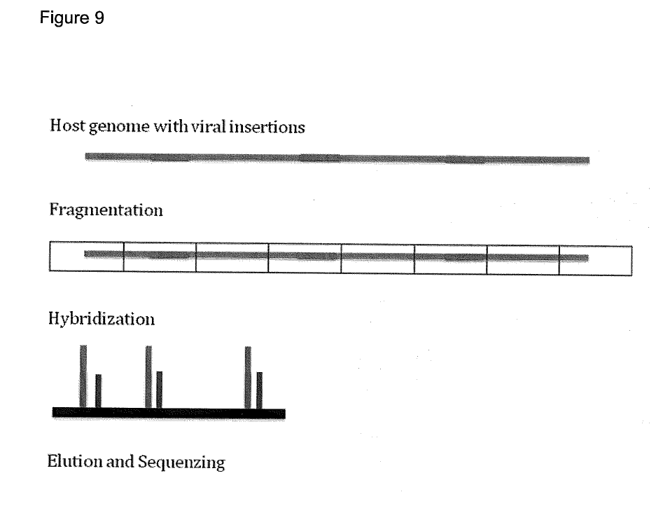

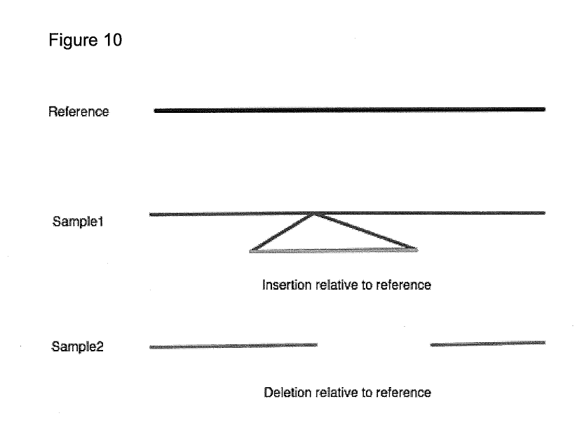

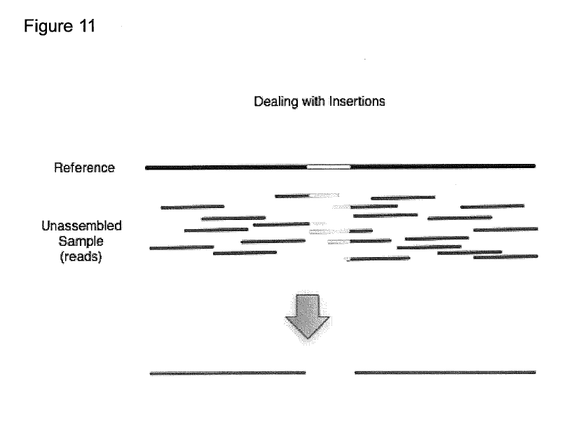

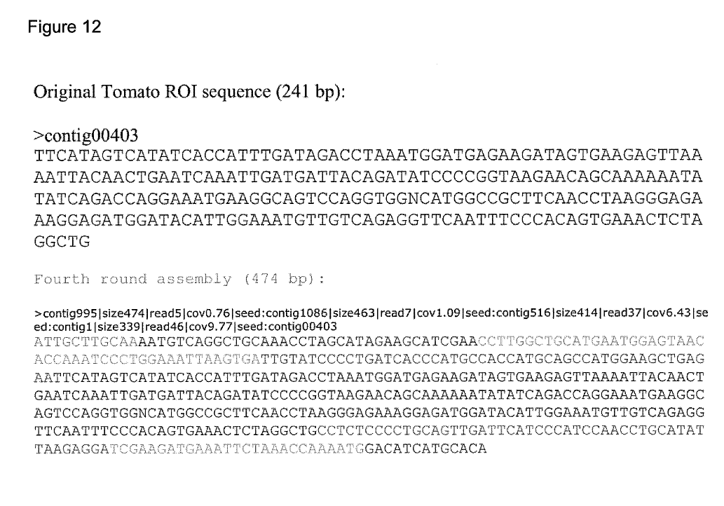

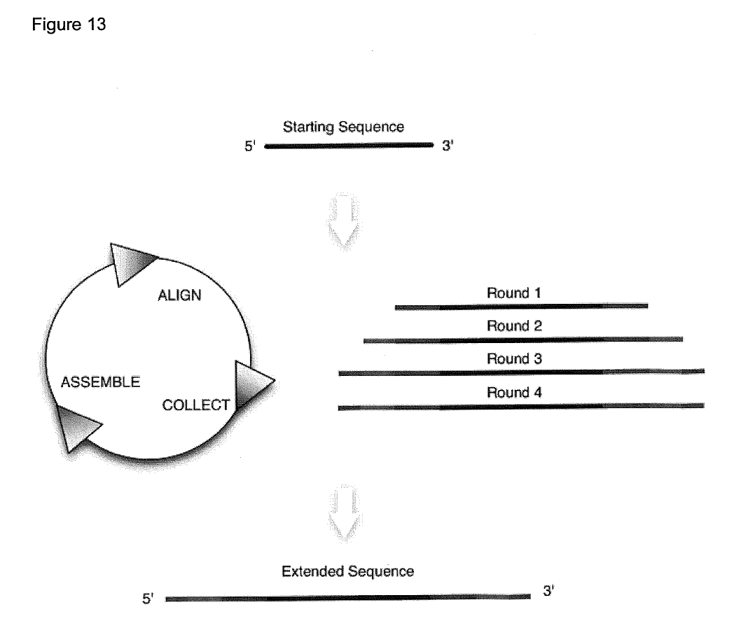

S6: Isolation of target molecules from a mixture of 2 nucleic acid populations: E. coli strain K12 in a mixture with human genomic DNA in the ratio of 1:750 (2 ng/1,500 ng)—isolation of parts of the nucleic acid population of E. coli K12. Probes which are complementary to sequences from E. coli K12 are used as capture probes. Detailed identification of the nucleic acid population isolated by subsequent sequencing.

S3: Isolation of target molecules from 1 nucleic acid population:

E. coli strain K12 (2 ng)—isolation of parts of the nucleic acid population of E. coli K12. Probes which are complementary to sequences from E. coli K12 are used as capture probes. Detailed identification of the nucleic acid population isolated by subsequent sequencing.

Comparison of S6 (2 nucleic acid populations) with S3 (1 nucleic acid population): Increasing the complexity of the sample (addition of a further nucleic acid population) increases the performance of the isolation (enrichment) of the desired nucleic acid regions.

(S6 and S3: sequence analysis via Illumina Genome Analyzer)

FIG. 2:

Isolation of target molecules from a mixture of 3 nucleic acid populations: E. coli strain K12 in a mixture with pathogenic E. coli strain O157 in the ratio of 1:1,000 (O157:1 ng/K12:1,000 ng) plus 1,500 ng of human genomic DNA-isolation of parts of the nucleic acid population of O157. Probes which are complementary to sequences from E. coli O157 are used as capture probes. Detailed identification of the pathogen by subsequent sequencing.

The following types of capture probes are used:
Specific for O157: 7,546 capture probes
Common: 7,546 capture probes

The common capture probes are common to several E. coli strains (e.g. O157, K12).

At the bottom the sequencing result on the Illumina NGS platform is shown.

FIG. 3:

Consecutive isolation of human genes (BRCA1, BRCA2, TP53, KRAS) from a complex mixture of 3 nucleic acid populations (human genomic DNA, tRNA, herring sperm DNA) with two different capture probe sets. Two consecutive isolations are effected. The sequence analysis of TP53 is visualized.

### Top:

Reference sequence: TP53
Capture probes are combined to a probe consensus sequence; the sequence sections formed in this way are to be isolated from the nucleic acid population.

### Middle:

Sequence analysis of the 2nd cycle of the isolation of TP53 sequence sections (the reads of the sequence analysis are mapped on the probe consensus sequence formed from the capture probes); a considerably higher performance of the isolation compared with cycle 1 can be clearly seen; capture probes of isolation cycle 2 were different to capture probes from cycle 1.

### Bottom:

Sequence analysis of the 1st cycle of the isolation of TP53 sequence section; a lower performance of the isolation than in cycle 2 can be clearly seen; capture probes of isolation cycle 1 were different to capture probes from cycle 2

### (Cycle 1 and 2: Sequence Analysis Via Illumina Genome Analyzer)

FIG. 4:

Sample preparation for the enrichment of DNA fragments for subsequent sequence analysis by means of Roche/454 sequencing.

FIG. 5:

Consecutive isolation of human genes (BRCA1, BRCA2, TP53, KRAS) from a complex mixture of 3 nucleic acid populations (human genomic DNA, tRNA, herring sperm DNA) with two identical capture probe sets. Two consecutive isolations are effected. The sequence analysis of TP53 is visualized.

### Top:

Reference sequence: (region of interest): TP53
Capture probes are combined to a probe consensus sequence; the sequence sections formed in this way are to be isolated from the nucleic acid population.

### Middle:

Sequence analysis of the 1st cycle of the isolation of TP53 sequence sections (the reads of the sequence analysis are mapped on the regions of the capture probes); a considerably higher performance of the isolation compared with cycle 1 can be clearly seen; capture probes of isolation cycle 2 were identical to capture probes from cycle 1.

### Bottom:

Sequence analysis of the 2nd cycle of the isolation of TP53 sequence section; a lower performance of the isolation than in cycle 2 can be clearly seen; capture probes of isolation cycle 1 were different to capture probes from cycle 2

### (Cycle 1 and 2: Sequence Analysis Via Illumina Genome Analyzer)

A: The degree of increase in performance can be clearly seen with the aid of the scale (1st cycle: 16, 2nd cycle: 401). The scale unit is the so-called coverage, which indicates how often the corresponding base position is covered by sequence reads.

B, D: The comparison between the 1st and 2nd cycle shows that the sequence coverage in the 2nd cycle is considerably more homogeneous, and an effective homogenization was therefore achieved.

C, F: The comparison between the 1st and 2nd cycle shows that it was possible for sequence gaps which were still present in the 1st cycle to be effectively closed very effectively.

E: The comparison between the 1st and 2nd cycle shows that it was possible to increase the sensitivity of the sequencer, since in the 2nd cycle it was possible to analyze sequence sections which have fallen below the detection limit of the sequencer in the first cycle.

FIG. 6:

Consecutive isolation of human genes (BRCA1, BRCA2, TP53, KRAS) from a complex mixture of nucleic acid populations with 2 identical capture probe sets. 2 consecutive isolations are effected. The sequence analysis of a section of BRCA2 is visualized in detail.

### Top:

Reference sequence: (region of interest): BRCA2
Capture probes are combined to a probe consensus sequence; the sequence sections formed in this way are to be isolated from the nucleic acid population.

### Middle:

Sequence analysis of the 1st cycle of the isolation of BRCA2 sequence sections (the reads of the sequence analysis are mapped on those from the capture probes); a considerably higher performance of the isolation compared with cycle 1 can be clearly seen; capture probes of isolation cycle 2 were identical to capture probes from cycle 1.

### Bottom:

Sequence analysis of the 2nd cycle of the isolation of TP53 sequence section; a lower performance of the isolation than in cycle 2 can be clearly seen; capture probes of isolation cycle 1 were different to capture probes from cycle 2.

### (Cycle 1 and 2: Sequence Analysis Via Illumina Genome Analyzer)

A, B: The comparison between the 1st and 2nd cycle shows that it was possible for sequence gaps which were still present in the 1st cycle to be effectively closed very effectively.

FIG. 7:

Multi-cycle Isolation of nucleic acid populations employing a bead-based sequence capture matrix:

Low-complexity regions are removed from the nucleic acid population to be analyzed by binding to cotDNA-bound beads. The nucleic acid population is thereby enriched for high-complexity regions.

FIG. 8:

Multi-cycle Isolation of nucleic acid populations employing an agarose- or sepharose-based sequence capture matrix:

Low-complexity regions are removed from the nucleic acid population to be analyzed by binding to cotDNA-bound flow-through columns. The nucleic acid population is thereby enriched for high-complexity regions.

FIG. 9:

Schematic depiction of a protocol for the detection of viral integration sites in a host genome:

Integration of the LTR region of foamy virus into Mus musculus.

In this example, the detection of the vector integration into the target cell DNA was conducted via microarray-based enrichment of the viral LTR sequences and subsequent next generation sequencing of the integration site library (Illumina, paired-end sequencing).

Wild-type CD117+/ckit+ primitive hematopoietic cells were enriched from murine bone marrow and then transduced on RetroNectin CH296-coated plates with a foamy viral vector expressing the EGFP cDNA off an internal SFFV promoter (multiplicity of infection (MOI) ratio: 20 viral particles per cell). The next day, cells were harvested and transplanted i.v. into lethally irradiated syngenic recipient mice. 8 months post transplantation, mice were sacrificed and DNA from bone marrow and spleen of the mice was obtained. From the individual mouse analyzed here, the spleen DNA was processed to a fragment library according to the manufacturer's protocol (Illumina, paired-end DNA fragment-library). Herring sperm and tRNA-nucleic acid populations were added to form a complex mixture of nucleic acid populations and incubated with a microarray that contained capture probes that were designed to bind both, foamy viral and lentiviral vector-specific DNA sequences as well as sequences for the transgene and negative control sequences. Unbound and non-specific DNA fragments were removed by standard wash steps and the bound fragments were eluted by use of aqueous formamide. The eluate was evaporated and the remaining DNA was amplified by PCR for 10 cycles. The resulting amplified DNA fragments were subjected to a second cycle of enrichment on a microarray that contained the identical capture probes as in the first enrichment cycle. Washing and eluation was conducted as in the first enrichment cycle. The eluated DNA was amplified by means of PCR for 10 cycles before it was subjected to next generation sequencing on the Illumina machine. Due to the use of a paired-end sequencing approach, it was possible to map the proviral sequences that were enriched by 2 cycles of microarray-based enrichment to the host genome (Mus musculus). By bioinformatic analysis, 22 foamy viral integration sites were detected in the spleen DNA of Mus musculus, of which 12 were confirmed by classical methods on the same DNA (LM-PCR and subsequent pyrosequencing on a Roche 454 machine), while 10 were not found by these standard methods.

Sequences Mapped Against Mus_{—}musculus, ENS52.NCBI37

| | Integrationsite analysis | confirmed with LAM-PCR |
|---|---|---|
| Chromosome | with enrichment method | and 454 pyrosequencing |
| 1 | 71148208 | 71148208 |
| | 71148211 | 71148211 |
| | 71148494 | 71148494 |
| | 71148498 | 71148498 |
| | 71148499 | 71148499 |
| | 88258299 | 88258299 |
| | 88258301 | 88258301 |
| | 186237613 | |
| 10 | 20936786 | |
| | 8776473 | 8776473 |
| | 63406220 | |
| 13 | 107037356 | 107037356 |
| 17 | 21519360 | 21519360 |
| 19 | 13379930 | |
| | 16641858 | |
| 2 | 11099720 | 11099720 |
| | 122528643 | 122528643 |
| 4 | 94644112 | |
| 5 | 16282472 | |
| | 75715977 | 75715977 |
| | 75715979 | 75715979 |
| | 75715983 | 75715983 |
| | 75715984 | 75715984 |
| 6 | 4817592 | |
| | 69202114 | |
| 7 | 75273373 | 75273373 |
| | 75273385 | 75273385 |
| 8 | 125837183 | |
| 9 | 62674579 | 62674579 |

FIG. 10:

Next generation sequencing: Comparison to Reference

FIG. 11:

Next generation sequencing: Dealing with insertions

FIG. 12:

Recursive Walking: Walking into Flanking Regions

FIG. 13:

Next generation sequencing: Recursive Walking

1. A method for isolation of target nucleic acid molecules comprising the steps:
(a) providing a mixture of at least two populations of nucleic acid molecules,
(b) bringing the mixture into contact with a population of nucleic acid capture molecules under conditions under which target nucleic acid molecules from at least one of the populations can bind specifically to the capture molecules,
(c) separating off material not bound to capture molecules and
(d) isolating and optionally characterizing the target nucleic acid molecules isolated.

2. The method as claimed in claim 1, characterized in that the at least two nucleic acid populations originate from the same or different species.

3. The method as claimed in claim 1, characterized in that the at least two nucleic acid populations originate from different organisms of a species.

4. The method as claimed in claim 1, characterized in that the capture molecules are immobilized on a solid phase, e.g. an array, on particles or on a membrane.

5. The method as claimed in claim 1, characterized in that the capture molecules are present in the free form.

6. The method as claimed in claim 1, characterized in that the sequence of the capture molecules is derived from a database or internet database which contains nucleic acid sequences of sequenced organisms.

7. The method as claimed in claim 1, characterized in that at least one of the nucleic acid molecule populations carries a marking, which after sequence analysis allows assignment of sequence data to a particular nucleic acid population.

8. The method as claimed in claim 1, characterized in that several nucleic acid populations carry a marking which allows assignment of the sequence data to a particular nucleic acid population after the sequence analysis.

9. The method as claimed in claim 7, characterized in that the marking comprises a detectable group.

10. The method as claimed in claim 7, characterized in that the marking comprises one or more terminal adaptor sequences which make an amplification of the target molecules isolated possible.

11. The method as claimed in claim 1, characterized in that a mixture of at least one marked nucleic acid population and at least one non-marked nucleic acid population is analyzed.

12. The method as claimed in claim 1, characterized in that the sequence of the nucleic acid target molecules in the nucleic acid populations to be analyzed is not yet known.

13. The method as claimed in claim 1, characterized in that it comprises several successive isolation cycles using identical or different capture molecule matrices.

14. The method as claimed in claim 1, characterized in that it comprises several successive isolation cycles using identical or different capture molecule matrices.

15. The method as claimed in claim 1, characterized in that the parts of the nucleic acid population which have been isolated are subjected to a subsequent sequence determination.

16. The method as claimed in claim 1, characterized in that not all the nucleic acid populations analyzed are represented by capture molecules.

17. The method as claimed in claim 1, characterized in that a DNA-binding protein, in particular a DNA-binding protein with a single-stranded DNA-dependent ATPase activity, such as, for example, RecA and optionally ATP, are added when the components are brought into contact.

18. The use of the method as claimed in claim 1 for the determination of medical, e.g. diagnostic or prognostic, parameters.

19. The use as claimed in claim 18 for analysis of alternative splicing, for analysis of exon junctions, for analysis of variations in the number of copies, for analysis of translocation in tumor diagnostics, for analysis of microbiomes or for detection of pathogens.

20. The use as claimed in claim 19 for the detection of insertion sites of viral sequences in a host genome.
